(19) United States
(12) Patent Application Publication
(10) Pub. No.: US 2008/0058848 A1
(43) Pub. Date: Mar. 6, 2008

(54) Endcap for a Sampling Device

(76) Inventors: Don Griffin, Kennesaw, GA (US); Jack Griffis, Decatur, GA (US)

Correspondence Address: GARDNER GROFF GREENWALD & VILLANUEVA. PC, 2018 POWERS FERRY ROAD, SUITE 800, ATLANTA, GA, 30339, US

(21) Appl. No.: 10/566,107

(22) PCT Filed: Jul. 28, 2004
(86) PCT No.: PCT/US04/24408

§ 371 Date: Jan. 26, 2006

Related U.S. Application Data
(60) Provisional application No. 60/490,477, filed on Jul. 28, 2003.

(51) Int. Cl. A61B 17/14; A61M 5/00

(52) U.S. Cl. 606/182; 604/110

(57) ABSTRACT

A tip portion or endcap for a lancing device having a non-circular opening to provide tension and pressure upon the patient's skin before and/or during lancing of the skin for sample collection. In example embodiments, the endcap is shaped to prevent the lancet body from sticking within the hole or passing through the hole. The endcap may also be shaped to allow it to be used at various lancing sites on the body without changing the endcap.

### CROSS-REFERENCE TO RELATED APPLICATIONS

This application claims priority to U.S. Provisional Patent Application Ser. No. 60/490,477, filed Jul. 28, 2003, which is hereby incorporated herein by reference in its entirety for all purposes.

### TECHNICAL FIELD

The present invention relates generally to medical devices and procedures, and more particularly to lancing devices. The invention also relates to an endcap for use in conjunction with a lancing device that allows for a collection of an increased volume of blood or bodily fluid to be obtained, and which reduces the sensation of pain.

### BACKGROUND OF THE INVENTION

Many medical procedures require puncturing of the skin, and sometimes underlying tissues, of an animal or human subject. For example, a sharp lancet tip is commonly used to puncture the subject's skin for sampling of blood or other body fluid, as for example in blood glucose monitoring by diabetics. Generally, lancets are much thinner than traditional hypodermic syringe needles; and therefore result in less pain to the patient.

However, one common problem that has been found to exist with utilizing thin lancets is the closure of the puncture site before an adequate volume of blood has been collected. Premature closure of the puncture requires additional blood to be drawn from the patient, often in a separate and sometimes less desirable location, resulting in an increase in pain and stress. Costs also increase, as multiple lancets are required to acquire an adequate sample.

Attempts have been made to provide lancing devices that inhibit premature closure of the puncture site. Such past attempts have included the use of an outer ring positioned away from the puncture site to “milk” or apply pressure around the wound to express a larger sample and prevent closure of the skin. Although utilizing the aforementioned circular ring often allows a larger sample to be collected before closure of the puncture site, occasionally a sample size is still insufficient, and another lancing operation to collect blood or analytic fluid is required.

It is also desirable to minimize the pain sensed by the subject resulting from the lancing and sampling procedure. Pain causes many subjects to avoid sampling or to sample less frequently than is desirable, sometimes leading to adverse health consequences. Lancing is commonly carried out on the subject's fingers, typically on the tips of the fingers, where capillary density is relatively high near the skin surface, to produce good sample size. But because nerve concentration is also high in these regions, fingertip lancing often results in considerable pain for many subjects.

Thus it can be seen that needs exist for improvements to lancing devices to provide a mechanism that allows for an increased volume of blood or other analytic fluid to be expressed and collected before the puncture site closes, while reducing the sensation of pain resulting from the sampling procedure. Needs further exist for such a mechanism that is readily adaptable to current lancing devices and procedures.

### SUMMARY OF THE INVENTION

In example forms, the present invention is an endcap for a lancing device that allows for an increased volume of blood or other body fluid to be collected from a lancing site on the skin of a human or animal subject. In example embodiments, the device compresses the skin's surface in a non-circular displacement region around the lancing site. In further embodiments, the device provides compression in a non-circular ring having a diameter smaller than the average outside diameter of the lancet, to prevent accidental discharge from or sticking of the lancet in the opening of the endcap.

The present invention enhances sample collection by preventing premature wound closure, and/or by applying tension and/or compression to the skin and underlying tissue at and around the sampling site to stimulate blood flow. Thus, a smaller (and consequently less painful) wound is required for collection of a designated sample size; or conversely, a larger sample can be collected from a wound of a given size. The enhanced sample collection provided by the present invention also facilitates sampling from alternate sites on the subject's body, such as the forearm, the earlobe, and/or other sites where nerve density (and thus resultant pain sensation) may be lower than at the fingertips. Contact by the irregular surface elements located about the periphery of the non-circular endcap opening of the present invention may also confuse the nerves at and around the sampling site, leading to a reduced sensation of pain from the sampling procedure.

In one aspect, the present invention is a lancing device for collecting a sample of body fluid from a sampling site on the skin of a subject. The lancing device preferably includes an outer body housing and a lancet, the lancet being movable between a first position within the outer body housing and a second position wherein at least a sharp tip portion of the lancet extends through an opening in the outer body housing. The opening in the outer body housing preferably has a non-circular periphery with a plurality of inwardly and outwardly directed lobes.

In another aspect, the invention is an endcap for a lancing device. The endcap preferably has a first end for connection to the lancing device, and a second end defining an opening through which a sharp tip of a lancet can pass. The opening preferably has a non-circular periphery including a plurality of inwardly and outwardly directed lobes.

In another aspect, the invention is an endcap for a lancing device shaped to allow the lancet to be used at alternative sites on different regions of the subject's body. In yet another aspect, the invention is an endcap sized and/or shaped to prevent blood from contacting the lancing device during collection.

In yet another aspect, the invention is a lancing device having a housing with an opening for passing a sharp lancet tip, the opening being lobed or non-circular, to interact with a greater surface area of skin surrounding the lancing site, for imparting pressure to milk a larger sample from the wound.

In another aspect, the invention is a method for acquiring an increased volume of analytic fluid from a sampling site. The method preferably includes providing a lancing device with an endcap defining a non-circular opening; placing the lancing device against the skin so that the skin is in communication with the non-circular opening; applying sufficient pressure on the lancing device to cause tension upon the skin within the non-circular opening; triggering the lancing device; and continuing to apply pressure upon the skin with the lancing device while fluid is extracted from the sampling site.

In still another aspect, the invention is a method of reducing the sensation of pain by a subject during a lancing procedure, said method comprising contacting a skin surface at or adjacent a sampling site with an irregular lobed contact surface.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 shows a partial cross sectional view of a lancing device having an endcap according to one example embodiment of the present invention.

FIG. 2 shows a perspective view of the endcap of FIG. 1.

FIG. 3 shows a front view of the endcap of FIG. 1.

FIG. 4 shows a perspective view of an endcap having a “star” shaped or multi-lobed opening in accordance with yet another example embodiment of the present invention.

FIG. 5 shows a front view of an endcap having a raised rim disposed around a non-circular opening according to another example embodiment of the present invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

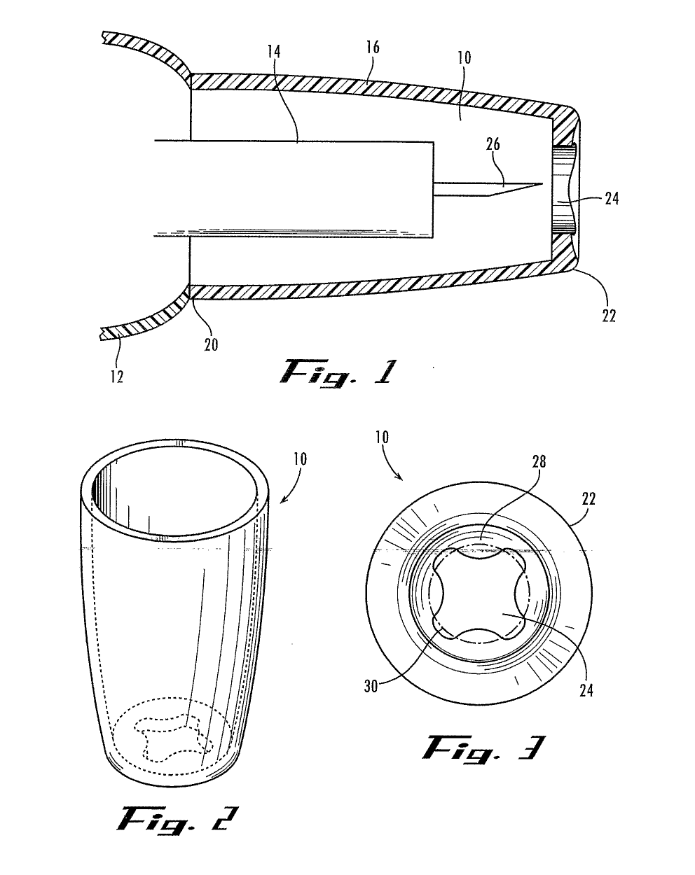

With reference now to the drawing figures, an endcap 10 for a lancing device 12 housing a lancet 14 therein is shown by way of example embodiments of the present invention. As shown in FIGS. 1-2, the endcap 10 preferably is formed of a substantially rigid body 16 having a first end 20 and a second end 22, wherein the first end 20 is sized and/or shaped to attach to the lancing device 12. The attachment of the endcap 10 to the lancing device 12 can be accomplished by way of a releasable or permanent coupling such as for example a threaded screw coupling, a socket, snap fittings, adhesive or welding, one or more screws or other connectors, or the like; or the endcap 10 can comprise an integral portion of the overall housing of the lancing device 12. In an example embodiment, at least a portion of the endcap 10 is formed of a transparent material to allow the patient and/or clinician to view the puncture site during lancing of the skin and collection of the bodily fluid, and thereby monitor the sample size.

As shown in FIGS. 2 and 3, the second end 22 of the body 16 defines a non-circular opening 24 through which a sharp tip 26, such as the tip of the lancet 14, projects to lance the skin of the subject and form a wound at the sample site from which a sample of body fluid is expressed and collected. The non-circular opening 24 is preferably defined by one or more inwardly directed lobes 28 extending from the second end 22 of the body 16. In further embodiments, additional lobes are used to further increase the surface area of the endcap that is applied to the skin. In still further embodiments, shapes which increase the tension of the endcap upon the skin are incorporated into the design. In one preferred form, the non-circular opening 24 is sized and/or shaped to have an inside diameter that is smaller than the average outside diameter of the lancet to prevent “sticking” of the lancet body within the hole or passage of the lancet body through the hole. In further embodiments, the opening 24 is shaped to prevent bodily fluids blood from contacting the body 16 of the endcap 10 during lancing and subsequent collection.

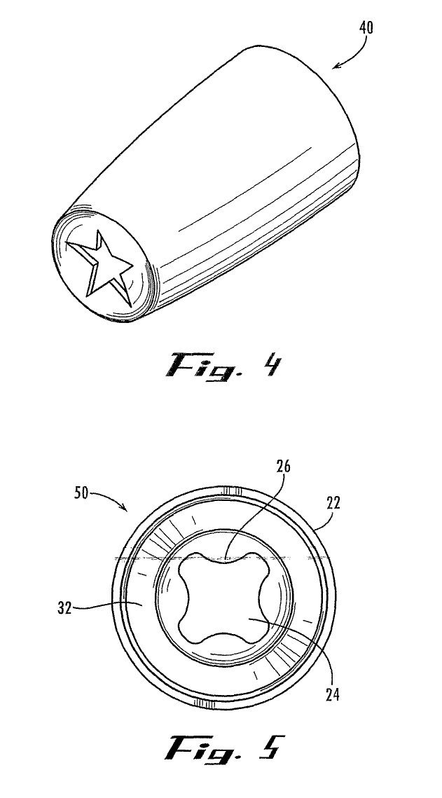

In various example embodiments, the opening 24 comprises an irregular or non-circular opening having one or more inwardly and/or outwardly projecting lobes or teeth for engaging the skin surrounding the lancing site. For example, and as seen best with reference to FIGS. 2 and 3, four inward lobes and four outward lobes are provided, relative to a reference circle 30 shown in broken lines. In the depicted embodiments, the lobes are rounded, having a smoothly-radiused curvature. In alternate embodiments, the lobes comprise one or more inwardly and/or outwardly projecting teeth having sharp angles rather than smooth curves. It will also be understood that, while the depicted embodiment comprises four inward lobes and four outward lobes, alternate forms of the invention comprise one, two, three or more lobes and/or teeth to define an irregular or non-circular opening. Indeed, there are many different shapes, sizes and configurations, and/or combinations thereof, such as an endcap 10 having a “star” shaped pattern having five inwardly and five outwardly projecting lobes, as depicted in FIG. 4, that are within the scope of the present invention, and will increase the surface area of tissue around the puncture site for contact and compression. It will also be understood that the invention encompasses a separate endcap for mounting to a lancing device, as well as a lancing device with an integral housing comprising an endcap portion thereof.

The lobes 28 can be planar (all within the same plane) or non-planar (having surface features projecting inwardly and/or outwardly relative to a reference plane coincident with the overall forward surface of the endcap or the lancing device housing). As depicted in FIG. 4, non-planar lobes can be provided to further increase the total skin surface area that can be captured and placed in tension and/or compression during lancing and to continually apply surface pressure or intermittently pump the area surrounding the lancing site following lancing.

In yet another embodiment, the opening 24 is surrounded by a raised rim 32, as depicted in FIG. 5, or one or more raised projections projecting outwardly from the body 16 to apply pressure upon the skin when lancing. In still another embodiment, the non-circular opening 24 is flat.

In operation, the non-circular opening 24 of the lancing device 12 is placed against the patient's skin, either at a fingertip or at an alternate lancing site. The patient or clinician then applies sufficient pressure on the lancing device 12 to impart pressure upon the skin within the non-circular opening, which creates an irregular compression zone. The patient then triggers the device 12, which causes the lancet 14 to pierce the skin, and continues to apply pressure upon the skin with the device 12 while fluid, such as blood, is extracted so that an increased volume of fluid is acquired. In alternate manners of operation, the lancing device is pumped, rocked and/or twisted with the irregular surface features surrounding the non-circular opening in contact with the skin surrounding the sampling site, thereby alternating points of tension and compression at the sampling site, to increase sample size and assist in preventing premature wound closure.

An unexpected advantage of the present invention may be a decreased sensation of pain by at least some subjects, believed to result from confusion or distraction of the nerves at and around the sampling site due to contact by the irregular lobed periphery of the opening against the skin surrounding the sampling site. This advantage may be enhanced in embodiments of the invention incorporating one or more outwardly-projecting, non-planar lobes with sharply-pointed distal tips for contact with the skin surface.

While the invention has been described with reference to preferred and example embodiments, it will be understood by those skilled in the art that a variety of modifications, additions and deletions are within the scope of the invention, as defined by the following claims.

1. A lancing device for collecting a sample of body fluid from a sampling site on the skin of a subject, said lancing device comprising an outer body housing and a lancet, the lancet being movable between a first position within the outer body housing and a second position wherein at least a sharp tip portion of the lancet extends through an opening in the outer body housing, and wherein the opening in the outer body housing has a non-circular periphery comprising a plurality of inwardly and outwardly directed lobes.

2. The lancing device of claim 1, wherein the periphery of the opening is non-planar.

3. The lancing device of claim 1, further comprising a raised rim around the opening.

4. The lancing device of claim 1, wherein the opening is generally cross-shaped, comprising four lobes.

5. The lancing device of claim 1, wherein the opening is generally star-shaped, comprising five lobes.

6. The lancing device of claim 1, wherein the lobes are smoothly-rounded.

7. The lancing device of claim 1, wherein the lobes comprise teeth with sharp points.

8. The lancing device of claim 7, comprising at least one outwardly-projecting, non-planar lobe having a sharply-pointed distal tip.

9. The lancing device of claim 1, wherein at least a portion of the outer body housing near the opening comprises a transparent material.

10. The lancing device of claim 1, wherein the opening has an inside dimension that is smaller than a corresponding outside dimension of the lancet, thereby preventing the lancet from becoming stuck in the opening.

11. An endcap for a lancing device, said endcap comprising a first end for connection to the lancing device, and a second end defining an opening through which a sharp tip of a lancet can pass, the opening having a non-circular periphery comprising a plurality of inwardly and outwardly directed lobes.

12. The endcap of claim 11, wherein the periphery of the opening is non-planar.

13. The endcap of claim 11, further comprising a raised rim around the opening.

14. The endcap of claim 11, wherein the opening is generally cross-shaped, comprising four lobes.

15. The endcap of claim 11, wherein the opening is generally star-shaped, comprising five lobes.

16. The endcap of claim 11, wherein the lobes are smoothly-rounded.

17. The endcap of claim 11, wherein the lobes comprise teeth with sharp points.

18. The endcap of claim 17, comprising at least one outwardly-projecting, non-planar lobe having a sharply-pointed distal tip.

19. The endcap of claim 11, further comprising a transparent portion near the opening.

20. A method for acquiring an increased volume of analytic fluid from a sampling site, the method comprising the steps of:
providing a lancing device with an endcap defining a non-circular opening;
placing the lancing device against the skin so that the skin is in communication with the non-circular opening;
applying sufficient pressure on the lancing device to cause tension upon the skin within the non-circular opening;
triggering the lancing device; and
continuing to apply pressure upon the skin with the lancing device while fluid is extracted from the sampling site.

21. (canceled)

22. A method of reducing the sensation of pain by a subject during a lancing procedure, said method comprising contacting a skin surface at or adjacent a sampling site with an irregular lobed contact surface, wherein the step of contacting a skin surface at or adjacent a sampling site with an irregular lobed contact surface comprises contacting the skin surface with at least one outwardly-projecting, non-planar lobe having a sharply-pointed distal tip.
